(19) United States
(12) Patent Application Publication
(10) Pub. No.: US 2004/0116498 A1
(43) Pub. Date: Jun. 17, 2004

(54) Regulations of lipids and/or bone density and compositions therefor

(76) Inventor: Alan James Husband, New South Wales (AU)

Correspondence Address: FINNEGAN, HENDERSON, FARABOW, GARRETT & DUNNER, LLP, 1300 I STREET, NW, WASHINGTON, DC, 20005, US

(21) Appl. No.: 10/250,858

(22) PCT Filed: Jan. 16, 2002
(86) PCT No.: PCT/AU02/00042

(30) Foreign Application Priority Data
Jan. 16, 2001 (AU) PR 2554

(51) Int. Cl. A61K 31/40; A61K 31/353

(52) U.S. Cl. 514/408; 514/456

(57) ABSTRACT

A method and compositions for regulating bone density and/or circulating lipid levels in a subject based on the combined administration of at least one isoflavone or functional derivative, equivalent or analogue thereof and at least one lipid regulating drug. The method and compositions are applicable to the beneficial alteration of blood lipoprotein levels, the improvement of vascular compliance, the decrease in the propensity of thrombogenic events, the reduction in the risk of vascular disease, coronary heart disease, and arteriosclerosis, and to the treatment or prevention of osteoporosis.

### FIELD OF THE INVENTION

The present invention relates to the regulation of lipids and/or bone density in mammals using lipid regulating drugs in combination with isoflavone compounds, and particularly but not exclusively to methods of treatment and/or prevention of osteoporosis and hyperlipidemia and compositions useful for same.

### BACKGROUND OF THE INVENTION

Cholesterol-lowering therapy has emerged as a mainstay in treating and preventing cardiovascular disease (CVD). Reducing elevated concentrations of low-density lipoprotein (LDL) cholesterol, or increasing high-density lipoprotein (HDL) cholesterol in patients with evidence of coronary heart disease (CHD), or with a family history of heart disease, may prevent strokes, heart attacks and reduce cardiac events as well as mortality in high-risk individuals [Sacks FM et al; Hebert PR et al; Scandinavian Simvastatin Survival Study Group; Parfitt].

However, many of the lipid regulating drugs have known deleterious side effects including: gastro-intestinal disturbances such as heartburn, epigastric pain, nausea, and diarrhoea; peripheral vasodilation resulting in flushing, itching, and a sensation of heat; headaches; dizziness; vertigo; fatigue; and skin rashes. In addition, reversible increases in serum-aminotransferase concentrations may occur and liver function should be monitored [Parfitt].

Estrogen Replacement Therapy (ERT) and Hormone Replacement Therapy (HRT) have been considered to have cardiovascular-protective abilities based on the relationship between decline in estrogen levels and adverse effects on blood lipoproteins, which are a risk factor of cardiovascular disease. However, there is evidence which disproves this theory. For example, a significant study conducted in the USA (known as the HERS study and involving 2763 women in a randomised, blinded, placebo-controlled secondary prevention trial), which set out to ascertain whether estrogen plus progestin HRT therapy provided a cardiovascular-protective effect, provides evidence that there is no overall cardiovascular benefit from HRT therapy and, in fact, that a trend towards early increase in the risk of coronary heart disease may result from HRT [Hulley et al JAMA 1998; and JAMA 1999].

ERT and HRT are also associated with many side effects including: nausea and vomiting; weight changes; breast enlargement and tenderness; pre-menstrual-like syndrome; fluid retention; changes in liver function; cholestatic jaundice; rashes and chloasma; depression; headache; and decreased contact lens tolerance. In addition, the use of estrogen without a progestogen results in endometrial hyperplasia and an increased risk of endometrial carcinoma. Furthermore, the current use of menopausal HRT is associated with an increased risk of venous thromboembolism, and long term use of HRT may be associated with an increased risk of breast cancer [Parfitt; Evans et al; Winship et al]. Indeed, the failure of the above-mentioned HERS study to prevent cardiovascular disease was mainly due to the induction of thromboembolic disease in women on HRT.

The Chicago Centre for Clinical Research has performed a study examining the efficacy, in lowering LDL cholesterol levels, of simvastatin and an HRT drug called Prempro (conjugated estrogens/medroxyprogesterone acetate tablets) alone and in combination. It was observed that 37% of women on combined therapy achieved a lowered LDL cholesterol, compared with 29% of those on simvastatin and 17% on HRT. Both therapies alone and in combination improved HDL-cholesterol levels by 4-13%, and reduced total cholesterol levels by 9-24% [Chicago Center for Clinical Research].

Conversely, a study by Sbarouni et al was conducted comparing HRT, simvastatin, and their combination, in the management of hypercholesterolemia in postmenopausal women. It was discovered that whilst all treatments were effective as compared to placebo (p<0.001), combination therapy was no more effective than statin treatment alone. Therefore no additional benefit was seen to be obtained through co-administration of statins with HRT.

Moreover, whilst the above-mentioned HRT/simvastatin treatment (the Chicago study) has been shown to lower cholesterol levels, the findings that long term HRT has no cardiovascular-protective effect (Hulley et al (1998)) may be considered to nullify the benefits of its use. Furthermore, as stated previously, the use of drugs such as simvastatin are associated with deleterious side effects, as are HRT or ERT agents.

In a related area of public health loss of bone density, like cardiovascular disease, is emerging as a major community health problem in Western communities that are experiencing increasing longevity. Loss of bone density appears to be associated primarily with declining estrogen levels in the body.

Recently Chan et al reported that statins increase bone mineral density in humans and thereby decrease the risk of osteoporotic fractures. However, these drugs have already been identified as possessing many undesirable side effects, which limits their benefits. Accordingly, there may be considered a need to identify methods and compositions for the effective and safe regulation of bone density in animals.

Research has been performed on the effect of the phytoestrogenic isoflavones on lipoprotein levels and on bone density. This interest stems from the fact that isoflavones exhibit estrogenic activity that may provide cardiovascular-protective qualities, and the epidemiological observations that cardiovascular diseases are less common in communities whose diets are rich in isoflavones.

However, studies conducted in which subjects were fed dietary supplements that were highly enriched for isoflavones failed to find any significant effects of the dietary supplementation on LDL or HDL levels [Nestel et al (1997 & 1999); Hodgson et al; Samman et al]. Further research in this area has been conducted by Potter et al (1998) [also reported in U.S. Pat. No. 5,855,892] using soy powder and soy protein products. These products did produce an effect on the HDL and LDL levels, however, they contained a wide variety of soy components such as saponins and sterols, which have known cholesterol-modifying properties. Further, such products are well known to modify dietary habits through the weight of protein present therein. Accordingly, no direct relationship between isoflavone consumption and lipid modulation may be deduced.

Several researchers have studied the effects of isoflavones obtained from red clover on cholesterol levels [Baber et al; Clifton-Bligh et al; Woodside et al]. These studies found that the isoflavones, when administered alone, may increase HDL-cholesterol levels and the HDL:LDL ratio; increased HDL-cholesterol levels have been linked to reduced risk of coronary heart disease. Additionally, the administration of isoflavones to subjects has been reported to increase bone density therein.

In respect of the effects of the isoflavones on osteoporosis, reports are sparse. U.S. Pat. No. 5,424,331 provides one report. In this patent it is claimed that a composition containing isoflavones (genistein and daidzein), in combination with an extensive mixture of specific compounds, may be beneficial for the prevention and/or treatment of osteoporosis in humans. However, U.S. Pat. No. 5,424,331 makes no mention of the beneficial use of isoflavones other than genistein and daidzein, the effect of a particular isoflavone ratio, the beneficial effect of isoflavones alone, or the relative effect of the isoflavones either alone or in combination with other materials on bone.

Accordingly, it may be considered that there is a need to develop a method of regulating circulating lipid levels, particularly a method of lowering circulating LDL cholesterol levels, and a method of regulating bone density which methods preferably are not associated with the severity or number of side effects from known methods.

Thus the present invention seeks to provide an improved method of regulating circulating lipid levels, and/or of regulating bone density in animals or at least to provide the public with a useful choice. The present invention further seeks to provide pharmaceutical compositions useful in said methods.

### STATEMENT OF INVENTION

In accordance with the present invention it has been discovered that the combined administration of isoflavones and lipid regulating drugs aids in the regulation of circulating lipid levels. Through enhanced improvement of the LDL:HDL ratio, improved arterial compliance, and decreased propensity of thrombogenic events, such combined administration concomitantly enhances the cardiovascular-protective effects of the isoflavones. Further the inventor has discovered that the combined administration of isoflavones with lipid regulating drugs aids in the regulation of bone density, and accordingly has application in the treatment and/or prevention of osteoporosis.

In particular the inventors have unexpectedly found that the combined administration of isoflavones and statins leads to a higher elevation in HDL, or lower depression in LDL, and increased bone density, than with either isoflavones or statins alone. This combined treatment is believed to be particularly important for men, where isoflavones may not have the same magnitude of effect on cholesterol levels as in women, because of their lower sensitivity to oestrogen-driven changes in metabolism.

Accordingly, in one broad aspect of the present invention there is provided a method for regulating circulating lipids levels and/or regulating bone density comprising the step of administering to a subject an effective amount of at least one isoflavone or functional derivative, equivalent, or analogue thereof in combination with at least one lipid regulating drug.

In another aspect of the present invention there is provided a method for the treatment of osteoporosis and/or hyperlipidemia comprising the step of:

administering to a subject a pharmaceutically effective amount of at least one isoflavone or functional derivative, equivalent or analogue thereof and at least one lipid lowering drug.

In a further aspect of the present invention there is provided a pharmaceutical composition comprising at least one isoflavone, or functional derivative, equivalent, or analogue thereof of general formula I and at least one lipid regulating drug.

In another aspect of the present invention there is provided use of at least one isoflavone or functional derivative, equivalent or analogue thereof of general formula I, at least one lipid regulating drug and a pharmaceutically acceptable carrier and/or diluent in the manufacture of a pharmaceutical composition.

In a further aspect of the present invention there is provided use of at least one isoflavone or functional derivative, equivalent or analogue thereof of general formula I and at least one lipid regulating drug in the manufacture of a medicament for regulating circulating lipids levels and/or regulating bone density.

In another aspect of the present invention there is provided use of at least one isoflavone or functional derivative, equivalent or analogue thereof of general formula I and at least one lipid regulating drug in the manufacture of a medicament for the treatment of osteoporosis, hyperlipidemia, thrombogenic events, vascular disease, ischaemic heart disease, coronary heart disease and/or arteriosclerosis.

In a further aspect of the present invention there is provided a pharmaceutical agent for regulating circulating lipids levels and/or regulating bone density, said agent comprising at least one isoflavone or functional derivative, equivalent or analogue thereof of general formula I and at least one lipid regulating drug.

In another aspect of the present invention there is provided a pharmaceutical agent for the treatment of osteoporosis and/or hyperlipidemia, said agent comprising at least one isoflavone or functional derivative, equivalent or analogue thereof of general formula I and at least one lipid regulating drug.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### DETAILED DESCRIPTION OF THE INVENTION

These and other aspects of the present invention, which should be considered in all its novel aspects, will become apparent from the following general description. The invention will further be elucidated by the non-limiting example herein described.

The present invention provides a method for regulating circulating lipid levels and/or regulating bone density, in animals. Generally, the method involves the combined administration of an effective amount of at least one lipid regulating drug with at least one isoflavone or functional derivative, equivalent or analogue thereof. An unexpected synergistic effect is observed when administration of these compounds is combined, compared with the effect either compound may exhibit on bone density regulation and/or circulating lipid level regulation when used alone.

As used herein, the term “regulating circulating lipid levels” refers to both the up-regulation and down-regulation of certain lipids circulating in the blood of a subject. It will be appreciated that the up-regulation of particular circulating lipids may occur simultaneously with the down-regulation of other such circulating lipids; for example, the simultaneous up-regulation in HDL cholesterol levels and down-regulation in LDL cholesterol levels, concomitantly increasing the HDL:LDL ratio.

As used herein, the term “regulating bone density” will be understood to include the up-regulation, down-regulation, or maintenance of bone density. The invention is preferably applicable to the up-regulation of bone density, for example in cases of osteoporosis.

It will be appreciated by those of general skill in the art to which the invention relates teat the present invention is applicable to a variety of different animals. Accordingly, a “subject” includes any animal of interest. In particular the invention is applicable to mammals, including humans, cats, dogs, horses, deer, oxen, goats, sheep and cows.

“Combined administration”, as used herein, should be interpreted broadly. Accordingly, the term “combined administration” does not necessarily infer that the individual active compounds are contained within a single composition or formulation for administration to a subject as a single unit-dose. The invention contemplates that the individual active compounds may reside in distinct formulations which may be combined, or mixed together, immediately prior to administration, or alternatively administered separately. Accordingly, it will be appreciated that the actives may be administered simultaneously or sequentially. Sequential administration does not necessarily infer that the distinct formulations are administered immediately one after the other, a period of time may exist therebetween. However, where such sequential administration occurs, it will be appreciated that there is a current biochemical and/or physiological effect within the subject, as a result of the administration of the first or initial active agent, when the second or later active agent is administered.

An “effective amount” of an active, as referred to herein means an amount necessary to at least partially attain a desired response.

Lipid regulating drugs are considered herein to be chemical substances cable of regulating lipid levels in the circulating blood of animals. Lipid regulating drugs suitable for use in accordance with the invention include: the statins; (3-hydroxy-3-methylglutaryl-coenzyme A (HMG-Co A)) reductase inhibitors; fibric acid derivatives and related compounds (the fibrates); bile-acid binding resins; derivatives of nicotinic acid; and the omega-3 triglycerides. Non-limiting examples of specific lipid regulating drugs contemplated for use in the invention are provided in Table 1 below. It will be appreciated that the list is merely given by way of example and should not be considered to be exhaustive.

TABLE 1

| Drug group | Typical Drugs |
|---|---|
| Statins | Atorvastatin |
| | Cerivastatin |
| | Fluvastatin |
| | Lovastatin |
| | Pravastatin |
| | Simvastatin |
| Bile-acid binding resins | Cholestyramine |
| | Colestipol |
| Fibric acid derivatives and related | Bezafibrate |
| compounds | Clofibrate |
| | Fenofibrate |
| | Gemfibrozil |
| Nicotinic Acid and derivatives | Acipimox |
| | Nicofuranose |
| | Nicotinic Acid |
| Miscellaneous drugs | Omega-3 marine triglycerides |

Isoflavone compounds or functional derivatives, equivalents or analogues thereof useful in the present invention are depicted by the general formula I:

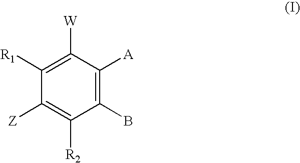

in which

R₁, R₂ and Z are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or

R₂ is as previously defined, and R₁ and Z taken together with the carbon atoms to which they are attached form a five-membered ring selected from

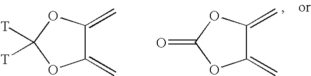

R₁ is as previously defined, and R₂ and Z taken together with the carbon atoms to which they are attached form a five-membered ring selected from

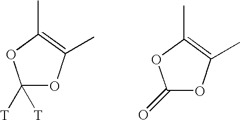

and

W is R₁, A is hydrogen, hydroxy, NR₃R₄ or thio, and B is selected from

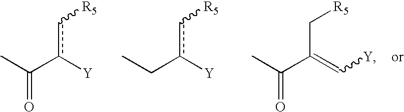

W is R₁, and A and B taken together with the carbon atoms to which they are attached form a six-membered ring selected from

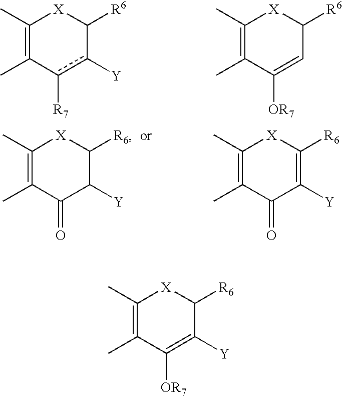

W, A and B taken together with the groups to which they are associated are selected from

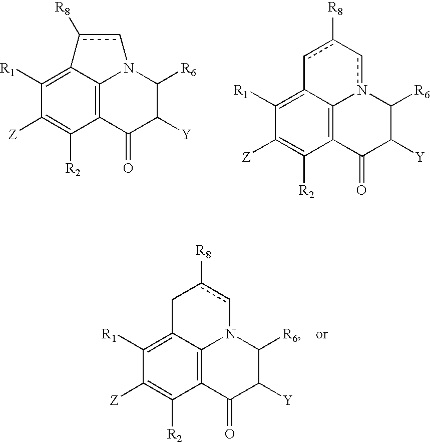

W and A taken together with the groups to which they are associated are selected from

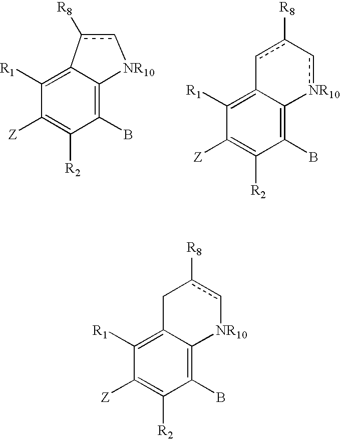

and B is selected from

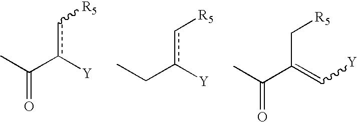

wherein

R₃ is hydrogen, alkyl, arylalkyl, alkenyl, aryl, an amino acid, C(O)R₁₁ where R₁₁ is hydrogen, alkyl, aryl, arylalkyl or an amino acid, or CO₂R₁₂ where R₁₂ is hydrogen, alkyl, haloalkyl, aryl or arylalkyl,

R₄ is hydrogen, alkyl or aryl,

or R₃ and R₄ taken together with the nitrogen to which they are attached comprise pyrrolidinyl or piperidinyl,

R₅ is hydrogen, C(O)R₁₁ where R₁₁ is as previously defined, or CO₂R₁₂ where R₁₂ is as previously defined,

R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,

R₇ is hydrogen, C(O)R₁₁ where R₁₁ is as previously defined, alkyl, haloalkyl, alkenyl, aryl, arylalkyl or Si(R₁₃)₃ where each R₁₃ is independently hydrogen, alkyl or aryl,

R₈ is hydrogen, hydroxy, alkoxy or alkyl,

R₉ is alkyl, haloalkyl, aryl, arylalkyl, C(O)R₁₁ where R₁₁ is as previously defined, or Si(R₁₃)₃ where R₁₃ is as previously defined,

R₁₀ is hydrogen, alkyl, haloalkyl, amino, aryl, arylalkyl, an amino acid, alkylamino or dialkylamino,

the drawing “═” represents either a single bond or a double bond,

T is independently hydrogen, alkyl or aryl,

X is O, NR₄or S, and

Y is

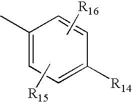

wherein

R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, thio, alkylthio, amino, alkylamino, dialkylamino, nitro or halo,

including pharmaceutically acceptable salts thereof.

Considered particularly useful in the present invention are compounds of the general formulae II-VIII:

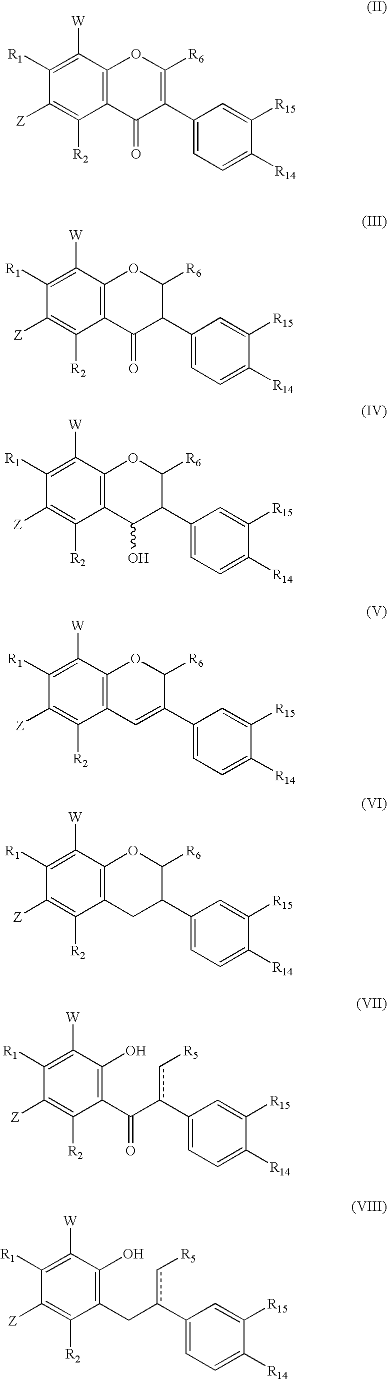

in which

R₁, R₂, R₅, R₆, R₁₄, R₁₅, W and Z are as defined above,

more preferably

R₁, R₂, R₁₄, R₁₅, W and Z are independently hydrogen, hydroxy, OR₉, OC(O)R₁₀, C(O)R₁₀, COOH, CO₂R₁₀, alkyl, haloalkyl, arylalkyl, aryl, thio, alkylthio, amino, alkylamino, dialkylamino, nitro or halo,

R₅ is hydrogen, C(O)R₁₁ where R₁₁ is hydrogen, alkyl, aryl, or an amino acid, or CO₂R₁₂ where R₁₂ is hydrogen, alkyl or aryl,

R₆ is hydrogen, hydroxy, alkyl, aryl, COR₁₁ where R₁₁ is as previously defined, or CO₂R₁₂ where R₁₂ is as previously defined,

R₉ is alkyl, haloalkyl, arylalkyl, or C(O)R₁₁ where R₁₁ is as previously defined, and

R₁₀ is hydrogen, alkyl, amino, aryl, an amino acid, alkylamino or dialkylamino,

more preferably

R₁ and R₁₄ are independently hydroxy, OR₉, OC(O)R₁₀ or halo,

R₂, R₁₅, W and Z are independently hydrogen, hydroxy, OR₉, OC(O)R₁₀, C(O)R₁₀, COOH, CO₂R₁₀, alkyl, haloalkyl, or halo,

R₅ is hydrogen, C(O)R₁₁ where R₁₁ is hydrogen or alkyl, or CO₂R₁₂ where R₁₂ is hydrogen or alkyl,

R₆ is hydrogen or hydroxy,

R₉ is alkyl, arylalkyl or C(O)R₁₁ where R₁₁ is as previously defined, and

R₁₀ is hydrogen or alkyl

and more preferably

R₁ and R14 are independently hydroxy, methoxy, benzyloxy, acetyloxy or chloro,

R₂, R₁₅, W and Z are independently hydrogen, hydroxy, methoxy, benzyloxy, acetyloxy, methyl, trifluoromethyl or chloro,

R₅ is hydrogen or CO₂R₁₂ where R₁₂ is hydrogen or methyl, and

R₆ is hydrogen.

Particularly preferred compounds of the present invention are selected from:

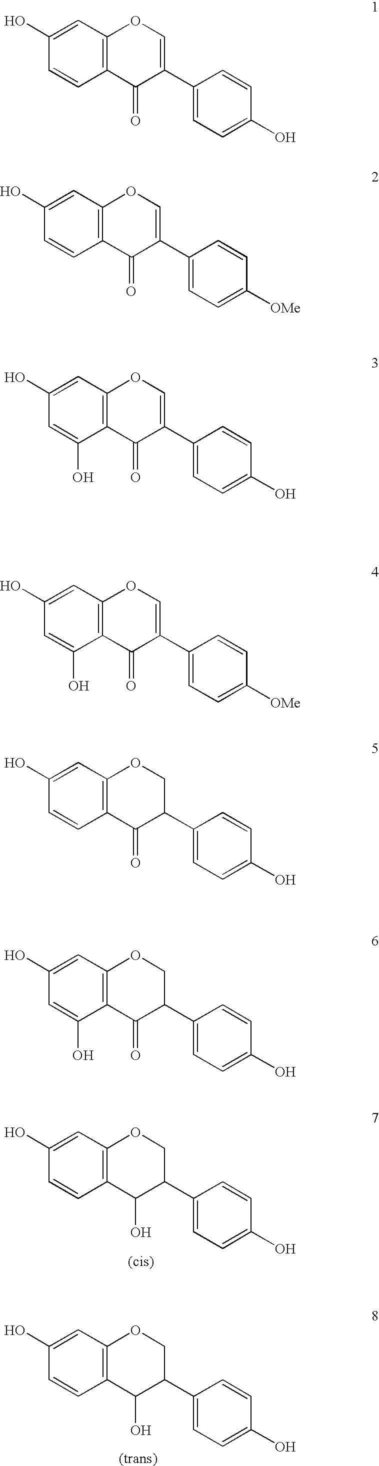

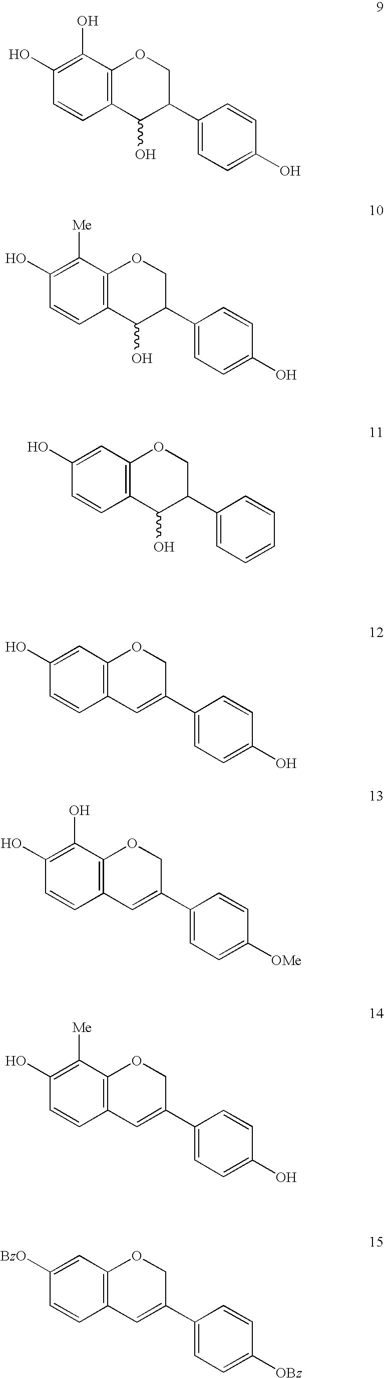

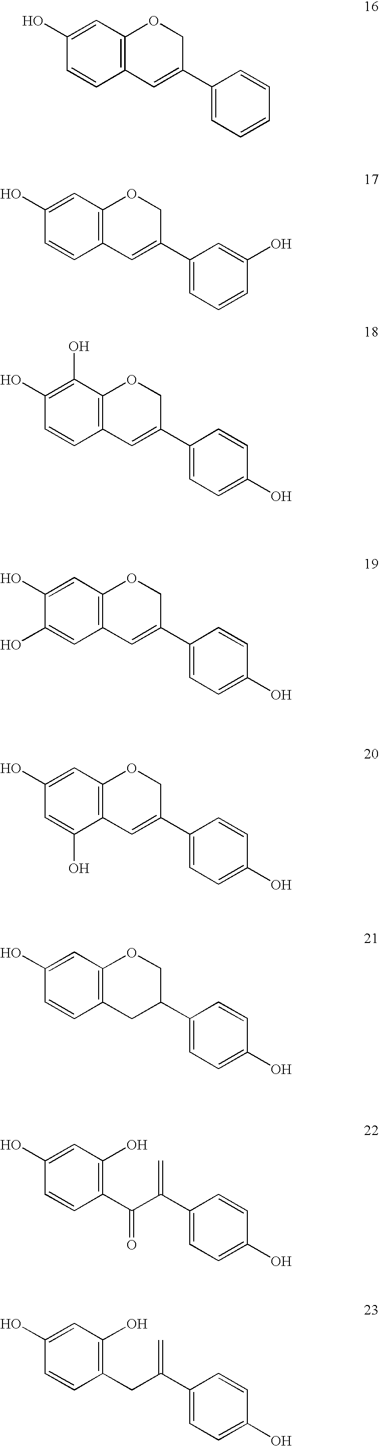

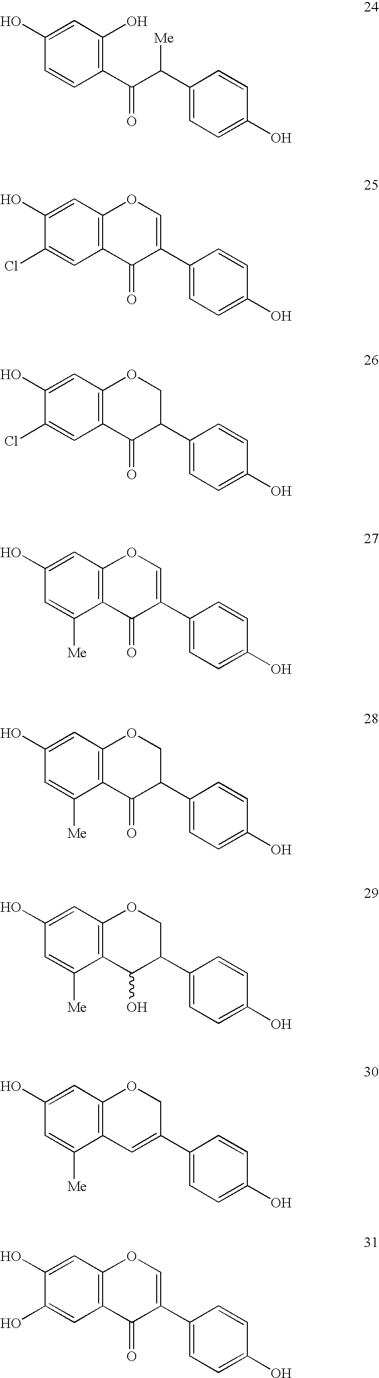

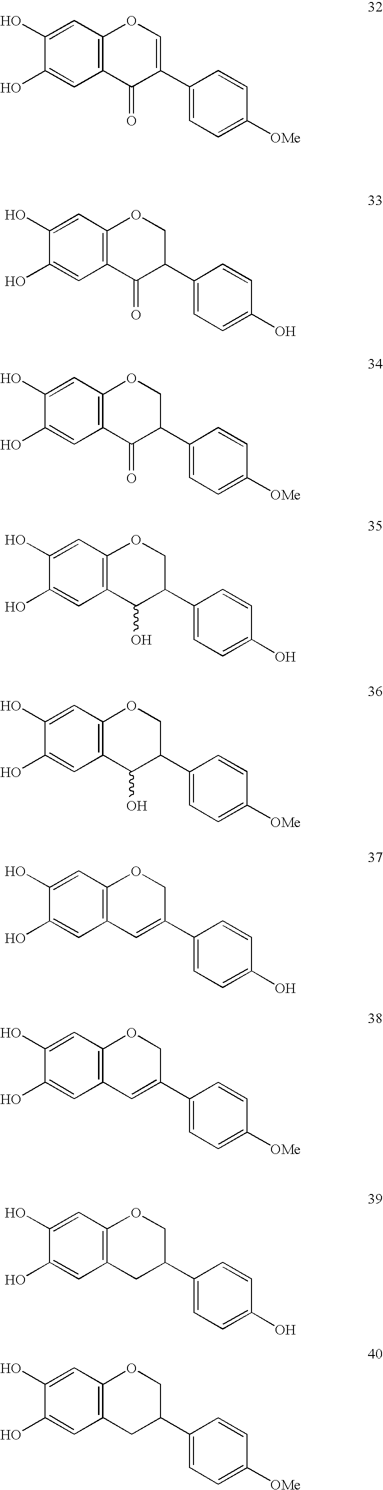

and pharmaceutically acceptable salts and derivatives having physiologically cleavable leaving groups thereof.

The preferred compounds of the present invention also include all derivatives with physiologically cleavable leaving groups that can be cleaved in vivo from the isoflavone molecule to which it is attached. The leaving groups include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted isoflavones and derivatives thereof are readily cleavable to the corresponding hydroxy substituted compounds.

Most preferred compounds contemplated for use in accordance with the invention are naturally occurring isoflavones and/or metabolites thereof including: formononetin, biochanin, genistein and daidzein.

As used herein, functional derivatives include metabolites, fragments, parts, portions and mutants from natural or synthetic isoflavone sources. It will be appreciated that such functional derivatives will exhibit one or more of the functional activities of the native isoflavone.

Similarly, chemical and functional equivalents of a particular isoflavone should be understood as molecules exhibiting any one of more of the functional activities of the isoflavone and may be derived from any source such as being chemically synthesised or identified via screening processes such as natural product screening.

Analogues of the isoflavones contemplated herein include, but are not limited to, those compounds depicted by general formula I.

The term “alkyl” is taken to include straight chain, branched chain and cyclic (in the case of 5 carbons or greater) saturated alkyl groups of 1 to 10 carbon atoms, preferably from 1 to 6 carbon atoms, such as methyl, ethyl, propyl, isopropyl, butyl, isobutyl, secbutyl, tertiary butyl, pentyl, cyclopentyl, and the like. The alkyl group is more preferably methyl, ethyl, propyl or isopropyl. The alkyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “alkenyl” is taken to include straight chain, branched chain and cyclic (in the case of 5 carbons or greater) hydrocarbons of 2 to 10 carbon atoms, preferably 2 to 6 carbon atoms, with at lease one double bond such as ethenyl, 1-propenyl, 2-propenyl, 1-butenyl, 2-butenyl, 2-methyl-1-peopenyl, 2-methyl-2-propenyl, and the like. The alkenyl group is more preferably ethenyl, 1-propenyl or 2-propenyl. The alkenyl groups may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl C₁-C₄-alkoxy, formyloxy, C₁-C₄-alkyl-carbonyloxy, C₁-C₄-alkylthio, C₃-C₆-cycloalkyl or phenyl.

The term “alkynyl” is taken to include both straight chain and branched chain hydrocarbons of 2 to 10 carbon atoms, preferably 2 to 6 carbon atoms, with at least one triple bond such as ethynyl, 1-propynyl, 2-propynyl, 1-butynyl, 2-butynyl, and the like. The alkynyl group is more preferably ethynyl, 1-propynyl or 2-propynyl. The alkynyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “aryl” is taken to include phenyl, biphenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy or halo.

The term “heteroaryl” is taken to include five-membered and six-membered rings which include at least one oxygen, sulfur or nitrogen in the ring, which rings may be optionally fused to other aryl or heteroaryl rings including but not limited to furyl, pyridyl, pyrimidyl, thienyl, imidazolyl, tetrazolyl, pyrazinyl, benzofuranyl, benzothiophenyl, quinolyl, isopuinolyl, purinyl, morpholinyl, oxazolyl, thiazolyl, pyrrolyl, xanthinyl, purine, thymine, cytosine, uracil, and isoxazolyl. The heteroaromatic group can be optionally substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl. The heteroaromatic can be partially or totally hydrogenated as desired.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The isoflavones for use in the present invention may be derived from any number of sources readily identifiable to a person skilled in the art. Preferably, they are obtained in the form of extracts from plant sources. Again, those skilled in the art will readily be able to identify suitable plant species, however, plants of particular use in the invention include chickpea and clover species, for example. More preferably, the isoflavone extract is obtained from red clover or subterranean clover species.

An isoflavone extract may be prepared by any number of techniques known in the art. For example, suitable isoflavone extracts may be prepared by water/organic solvent extraction from the native plant source. It will be appreciated that an isoflavone extract may be prepared from any single tissue of a single species of plant or a combination of two or more different tissues thereof. Similarly, an extract may be prepared from a starting material which contains a heterogeneous mixture of tissues from two or more different species of plant.

Generally, where an isoflavone extract is prepared from plant material, the material may be comminuted or chopped into smaller pieces, partially comnminuted or chopped into smaller pieces and contacted with water and an organic solvent, such as a water miscible organic solvent. Alternatively, the plant material is contacted with water and an organic solvent without any pre-treatment. The ratio of water to organic solvent may be generally in the range of 1:10 to 10:1 and may, for example, comprise equal proportions of water and solvent, or from 1% to 30% (v/v) organic solvent. Any organic solvent or a mixture of such solvents maybe used. The organic solvent may preferably be a C2-10, more preferably a C1-4 organic solvent (such as methanol, chloroform, ethanol, propanol, propylene glycol, erythrite, butanol, butanediol, acetonitrile, ethylene glycol, ethyl acetate, glycidol, glycerol dihydroxyacetone or acetone). Optionally the water/organic solvent mixture may include an enzyme which cleaves isoflavone glycosides to the aglycone form. The mixture may be vigorously agitated so as to form an emulsion. The temperature of the mix may range, for example, from an ambient temperature to boiling temperature. Exposure time may be between one hour to several weeks. One convenient extraction period is twenty-four hours at 90° C. The extract may be separated from undissolved plant material and the organic solvent removed, such as by distillation, rotary evaporation, or other standard procedures for solvent removal. The resultant extract containing water soluble and non-water soluble components may be dried to give an isoflavone-containing extract, which may be formulated with one or more pharmaceutically acceptable carriers, excipients and/or auxiliaries according to the invention.

An extract made according to the description provided in the previous paragraphs may contain small amounts of oil which include isoflavones in their aglycone form (referred to herein as isoflavones). This isoflavone enriched oil, may be subject to HPLC to adjust the isoflavone ratios, or, if it is at the desired isoflavone ratio, may be dried, for example in the presence of silica, and be formulated with one or more carriers, excipients and/or auxiliaries to give an isoflavone containing extract. Alternatively, the isoflavones contained in said small amounts of oil may be further concentrated by addition to the oil of a non-water soluble organic solvent such as hexane, heptane, octane acetone or a mixture of one or more of such solvents. One example is 80% hexane, 20% acetone w/w having high solubility for oils but low solubility for isoflavones. The oil readily partitions into thle organic solvent, and an enriched isoflavone containing extract falls out of solution. The recovered extract may be dried, for example in an oven at 50° C. to about 120° C., and formulated with one or more pharmaceutically acceptable carriers, excipients and/or auxiliaries.

It will be appreciated that the present invention also contemplates the production of compounds described herein, including the isoflavones, functional derivatives, equivalents, or analogues thereof, by established synthetic techniques well known in the art. See, for example, Chang et al which discloses methods appropriate for the synthesis of various isoflavones.

Other suitable methods may be found in, for example, published International Patent Applications WO 98/08503 and WO 00/49009, and references cited therein, which are incorporated herein in their entirety by reference.

It will be appreciated that isoflavones suitable for use in the present invention may be in the form of a powder, a slurry, in aqueous solution (for example, containing a small amount of oil), particulate form, or dissolved in an organic solvent (such as methanol, ethanol, ethyl acetate or dimethyl sulfoxide.

Based on the characteristic ability of the method and compositions of the present invention to regulate circulating lipid levels and bone density, they may be useful for the treatment and/or prevention of certain indications presenting in animals. Generally such indications include hyperlipidemias and osteoporosis. Specifically, the method and compositions are suitable for beneficially altering blood lipoprotein levels, improving vascular compliance and for the treatment and/or prevention of thrombogenic events, vascular disease, ischaemic heart disease, coronary heart disease, arteriosclerosis and Type I and II osteoporosis.

As used herein, the term “treatment” is to be considered in its broadest context. The term does not necessarily imply that an animal is treated until total recovery. Accordingly, “treatment” includes amelioration of the symptoms or severity of a particular condition or preventing or otherwise reducing the risk of developing a particular condition.

As previously mentioned, combined administration of the isoflavones and lipid regulating drugs according to the present invention may include the administration of two or more distinct compositions (each containing a different active according to the invention) or the administration of a single composition or formulation which contains the at least one isoflavone in combination with at least one lipid lowering drug.

In the case where the actives according to the invention are administered as distinct compositions each composition will contain either of at least one lipid regulating drug or at least one isoflavone or functional derivative, equivalent or analogue thereof, each optionally in association with one or more pharmaceutically acceptable carriers, excipients, auxiliaries and/or diluents.

In the case where the actives are combined in a single composition such composition preferably comprises at least one lipid regulating drug and at least one isoflavone or functional derivative, equivalent or analogue thereof, optionally in association with one or more pharmaceutically acceptable carriers, excipients, auxiliaries and/or diluents.

A “pharmaceutically acceptable carrier, excipient, auxiliary and/or diluent” as used herein should be taken to include any carrier, excipient, auxiliary or diluent that is considered useful in preparing a pharmaceutical composition. Such carriers, excipients, auxiliaries or diluents will be generally safe, non-toxic and neither biologically nor otherwise undesirable. The term also includes carriers, excipients, auxiliaries or diluents that are acceptable for veterinary use as well as human pharmaceutical use. As used herein the term “pharmaceutically acceptable carriers, excipients, auxiliaries and/or diluents” includes one of, or more than one of, such substances.

It will be appreciated that the carriers, excipients, auxiliaries or diluents used in the present invention may be in solid or liquid form, or both, depending on the desired dosage form of the composition, or compositions, to be used. Carriers, excipients, auxiliaries and/or diluents include flow ability agents, fillers, binders, stabilisers, antioxidants, lubricants/binders, disintegrants, and coatings. It will be appreciated that any number of carrier, excipient, auxiliary or diluent substances known in the art may be used in the invention. However, examples of suitable carriers, excipients, auxiliaries or diluents will become apparent in the ensuing paragraphs of this description.

In addition to the at least one lipid regulating drug and at least one isoflavone, the compositions of the invention may include one or more additional agents (or accessory ingredients), such as vitamins (for example, Vitamin A, Vitamin B group, Vitamin C, Vitamin D, Vitamin E and Vitamin K), and minerals (for example, magnesium, iron, zinc, calcium and manganese in the form of pharmaceutically acceptable salts).

The compositions of the invention include those suitable for oral, rectal, optical, buccal (for example, sublingual), parenteral (for example, subcutaneous, intramuscular, intradermal and intravenous injection) and transdermal administration. It will be appreciated that the most suitable route in any given case will depend on the nature and severity of the condition being treated, and the preference and general state of the patient.

Compositions suitable for oral administration may be presented in discrete units, such as capsules, sachets, lozenges, or tablets, each containing a pre-determined amount of the active combination. The actives may be present as powders or granules, as a solution or a suspension in an aqueous or non-aqueous liquid, or as an oil-in-water or water-in-oil emulsion, for example. Such compositions may be prepared by any suitable method of pharmacy which includes at least the step of bringing into association the active compound and one or more suitable carriers, auxiliaries, excipients, or diluents.

In general the oral compositions of the invention, which contain both the at least one lipid regulating drug and at least one isoflavone, are prepared by uniformly and intimately admixing the isoflavone/lipid regulating drug combination with a liquid or finely divided solid carrier, or both, and then, if necessary, shaping the resulting mixture. For example, a tablet may be prepared by compressing or moulding a powder or granules containing the extract, optionally with one or more accessory ingredients. Compressed tablets may be prepared by compressing in a suitable machine, the extracts in the form of a powder or granules optionally mixed with a binder, lubricant, inert diluents, and/or surface active/dispersing agent(s). Moulded tablets may be made by moulding, in a suitable machine, the powdered compound moistened with an inert liquid binder.

In the case of oral compositions, suitable carriers include fillers, binders and, if desired, disintegrators. Appropriate fillers include sugars, such as lactose, saccharose, mannitol or sorbitol, cellulose preparations and/or calcium phosphates, such as tricalcium phosphate or calcium hydrogen phosphate. Appropriate binders include, starch pastes comprising, for example, corn, wheat, rice or potato starch, gelatin, tragacanth, methylcellulose and/or polyvinylpyrrolidone. Appropriate disintegrators include, for example, the above-mentioned starches, carboxymethyl starch, cross linked polyvinylpyrrolidone, agar or algin acid or a salt thereof, such as sodium alginate. Excipients may include flow conditioners or lubricants; for example silicic acid, talc, stearic acid or salts thereof, such as magnesium or calcium stearate, and/or polyethylene glycol.

Dragee cores prepared according to the invention may be provided with suitable, optionally enteric coatings, there being used, inter alia, concentrated sugar solutions which may comprise gum Arabic, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or dragee coatings, for example, for identification purposes or to indicate different doses of active ingredients.

Other orally administrable pharmaceutical compositions are dry-filled capsules made, for example, of gelatine, and soft, sealed capsules made of gelatine and a plasticiser, such as glycerol or sorbitol. The dry-filled capsules may comprise the actives in the form of granules, for example, in admixture with fillers, such as lactose, binders, such as starches; and/or glicants, such as talc or magnesium stearate, and, where appropriate, stabilisers. In soft capsules, the actives are preferably dissolved or suspended in suitable liquids, such as fatty oils, paraffin oil or liquid polyethylene glycols, to which stabilisers may also be added.

Formulations suitable for buccal (sublingual) administration include: lozenges, comprising the actives in a flavoured base, for example, sucrose and acacia or tragacanth; and pastilles, comprising the actives in an inert base, for example, gelatine and glycerin, or sucrose and acacia.

Compositions of the present invention suitable for parenteral administration conveniently comprise sterile aqueous preparations containing the actives, which preparations are preferably isotonic with the blood of the intended recipient. These preparations are preferably administered intravenously, although administration may also be effected by means of subcutaneous, intramuscular, or intradermal injection. Suitable compositions include water soluble extracts and suspensions of the active ingredient. For example, oily injection suspensions may be used in which suitable lipophilic solvents or vehicles, such as fatty oils (for example sesame oil), or synthetic fatty acid esters (for example ethyl oleate or triglycerides), are used. Alternatively, aqueous injection suspensions may be utilised, which comprise viscosity-increasing substances, for example sodium carboxymethylcellulose, sorbitol and/or dextran, and, where appropriate, also stabilisers. As an example, compositions may conveniently be prepared by admixing the actives with water or a glycine buffer and rendering the resulting solution sterile and isotonic with the blood.

Formulations suitable for rectal administration are preferably presented as unit dose suppositories. These may be prepared by admixing the actives with one or more conventional solid carriers, for example cocoa butter, and then shaping the resulting mixture.

Formulations or compositions suitable for topical administration to the skin preferably tie the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 5% w/w, more particularly from 0.5% to 2% w/w. Examples of such compositions include cosmetic skin creams.

Compositions suitable for transdermal administration may be presented as discrete patches adapted to remain in intimate contact with the epidermis of the recipient for a prolonged period of time. Such patches may contain the actives in an optionally buffered aqueous solution.

Compositions suitable for transdermal administration may also be delivered by iontophoresis (see Panchagnula R, Pillai O, Nair V B, Ramarao P. Transdermal iontophoresis revisited. Curr Opin Chem Biol August 2000; 4(4):468-73) typically taking the form of an optionally buffered aqueous solution of the actives. Such compositions may, for example, contain citrate or bis/Tris buffer (pH 6) or ethanol/water, with for example 0.05% to 30% extract.

Formulations suitable for inhalation may be delivered as a spray composition in the form of a solution, suspension or emulsion. The inhalation spray composition may further comprise a pharmaceutically acceptable propellant such as carbon dioxide or nitrous oxide.

The compositions of the invention may also be administered to a human in a dietary supplement form. Dietary supplements incorporating the actives can be prepared by adding the composition to a food in the process of preparing the food. Any food may be used including, but not limited thereto, meats such as ground meats, emulsified meats and marinated meats; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; salad dressings; and dips and spreads such as mayonnaise, margarine, butter, butter substitute, and other fat containing spreads. The composition is added to the food in an amount selected to deliver a desired dose of the composition to the consumer of the food.

It will be appreciated that compositions suitable for use in the present invention may be prepared according to standard methodology used in the art. Such methodology is noted for example in Goodman & Gilman, The Pharmacological Basis of Therapeutics (7th Edition, 1985) and Remington's Pharmaceutical Science (Mack Publishing Company, 10th Edition.

Generally, compositions of the invention may be formulated to contain one or more isoflavone or functional derivative, equivalent or analogue thereof in the range of 1 mg to 1 g (total isoflavone content). Preferably, a composition according to the invention contains 10 mg to 160 mg of the isoflavones. Preferably, an administration at a rate of between 0.1 to 2 mg/kg body weight per day of total isoflavones is considered suitable for use in the present invention.

It is envisaged that where a composition of the invention is prepared using an isoflavone extract as described herein, such composition will generally contain between 0.5% to 100% by weight of the extract. In the case of injectable formulations, 0.1% to 60% w/v, is preferable. It will be appreciated that such percentage of extract to total composition may vary depending on the concentration of particular isoflavones contained within the extract and the desired unit dosage required in any particular composition.

A person of general skill in the art to which the invention relates will readily be able to determine the isoflavone content of an extract or composition according to the invention, or whether a particular ratio of one isoflavone to another exists therein, using techniques commonly used in the art; for example HPLC analysis. Similarly, such skilled person(s) will readily be able to produce a composition having such a desired isoflavone content and/or ratio by combining different isoflavone extracts, using HPLC fractionation, and the like.

Those of general skill in the art to which the present invention relates will understand that a composition according to the invention may be formulated to contain one or more lipid regulating drugs at a concentration conventionally used in the art. Such a person would be able to obtain such information from the manufacturers of such drugs.

While the preferred concentration ranges of actives in the compositions of the present invention have been described above, it will be appreciated that the amount of a particular active in a composition of the invention may be manipulated outside of these ranges to accommodate a desired dosage form and administration regime. It will also be appreciated that the preferred concentration of active compounds in any drug composition according to the invention, will depend on absorption, distribution, inactivation, and excretion rates of the drug as well as other factors known to those of skill in the art.

Compositions according to the invention may be administered in an effective amount, to a subject in need thereof, using varying regimes that may be tailored to the particular needs of a subject or patient. It will be appreciated that the administration regime, including the dose of actives administered per day, may vary, inter alia, according to the specific mode of administration, the nature and severity of the condition to be treated, the preference and state of health of the subject and based on the judgement of the health care provider.

Administration of the lipid lowering drugs is preferably at a rate recommended in the MIMS Annual [Caswell] and in this regard simvastatin, for example, may be administered at a dose of 10 mg per day, increasing to a dose of 40 mg per day.

The method of the present invention also contemplates the use of a low dose of a lipid regulating drug. As used herein “low dose” means a dosage lower than that described in the MIMS Annual; ie the concentration of such a drug which is administered to a subject is less than the medically documented dosage for the drug. For example, the recommended starting dose of a statin is between 10 mg and 20 mg daily. Accordingly, a low dose would be considered as a dose per day of less than 10 mg. By administering a low dose of such lipid regulating drugs, the occurrence and severity of associated side effects may be reduced whilst maintaining the beneficial action thereof, for example the lipid regulating action.

It will be appreciated that the desired daily dosage of each active may be administered as a single dose or as a number of divided doses.

The most preferred administration form is a solid dosage form such as a tablet or capsule. However, injectable formulations may also be used. Where this is the case, administration at a rate of 0.1 mL/minute/kg, for example, is considered appropriate.

The length of treatment of a particular subject according to the invention may vary depending on the specific mode of administration, the nature and severity of the condition to be treated, the preference and state of health of the subject and based on the judgement of the health care provider. Generally, an appropriate treatment length is one which is sufficient to at least partially attain a desired result. For example, in the case of circulating lipid regulation, administration may continue until such time as the circulating lipid levels are appropriate. However, it is considered that in order to obtain maximum benefit from the treatment (in relation to lipid ratios, vascular compliance, osteoporosis and decreased propensity to thrombogenic events, for example) administration may be long term, such as for one or more years.

The subjects in need of being treated using a method and compositions of the present invention include those animals, including mammals, particularly humans, who require a level of regulation of circulating lipid levels and/or bone density. For example, such subjects may be normocholesterolemic or hypercholesterolemic men or women who are artherosclerotic, or have low HDL, or men or women presenting symptoms of osteoporosis.

It will be appreciated that the combination of a particular lipid lowering drug and isoflavone or functional derivative, equivalent, or analogue thereof, according to the invention, may vary depending on the needs of a patient, whether a single unit dose containing both actives is formulated, and the method of formulation, for example. However, it is particularly preferred that where the treatment and/or prevention of osteoporatic indications is desired the lipid lowering drug be a statin.

The invention is now further elucidated with reference to the following non-limiting example.

### EXAMPLE 1

A forty-seven year old male presented with hypercholesterolemia. The male commenced taking 10 mg Lipitor (atorvastatin) simultaneously with Trinovin (red clover extract from Trifolium pratense which contains 40 mg standardised isoflavones per tablet) and found surprisingly, after 18 months, a synergistic therapeutic benefit from the Lipitor/Trinovin combination in lowering total cholesterol and LDL-cholesterol; compared with the use of either active alone, as previously reported.

| Months | | | |
|---|---|---|---|
| of treatment | | | |
| (Lipitor | Total cholesterol | LDL-cholesterol | HDL-cholesterol |
| and Trinovin) | (mmol/L) | (mmol/L) | (mmol/L) |
| 0 | 6.4 | 4.8 | 1.2 |
| 10 | 6.6 | 5.0 | 1.1 |
| 15 | 4.5 | 2.8 | 1.2 |

From the description provided herein it will be apparent that a synergistic therapeutic benefit may be obtained from the combined administration to a subject of at least one lipid regulating drug and at least one isoflavone. Accordingly, the invention may have the advantage that lower doses of each active, particularly the lipid regulating drugs, may be required to obtain a beneficial result. In this regard, the method of the invention may lower the chances of the subject suffering the undesirable side effects commonly associated with the use of such drugs. Similarly, it may mean that a subject, unable to use treatment methods based on either active, due to intolerance to the isoflavones and/or lipid lowering drugs for example, may tolerate and benefit from such a treatment regime.

The invention has been described herein, with reference to certain preferred embodiments, in order to enable the reader to practice the invention without undue experimentation. However, a person having ordinary skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

Bibliographic details of the publications referred to herein are collected at the end of the description.

The reference to any prior art in this specification is not, and should not be taken as, an acknowledgment or any form of suggestion that that prior art forms part of the common general knowledge in the field of endeavour.

Throughout this specification and the claims which, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### REFERENCES

Baber R. Clifton-Bligh P, Fulcher G, Lieberman D, Nery L, Moreton T The effect of an isoflavone dietary supplement (Rimostil) on serum lipids, forearm bone density and endometrial thickness in post-menopausal women. Proc. 10^{th} Annual Meeting of the North American Menopause Society, New York, 23-25 Sep. 1999.

Caswell A (ed) 1999 MIMS Annual, 23^{rd} edition, Singapore

Chan K A, Andrade S E, Boles M, Buist D S, Chase G A, Donahue J G, Goodman M J, Gurwitz, J H, Lacroix A Z, Platt R Inhibitors of hydroxymethylglutaryl-coenzyme A reductase and risk of fracture among older women. Lancet Jun. 24, 2000; 355(9222): 2185-8

Chang Y- C, Nair M G, Santell R C, Helferich W G Microwave-Mediated Synthesis of Anticarcinogenic Isoflavones from Soybeans J Agric Food Chem 1994, 42: 1869-1871

Chicago Center for Clinical Research—Company Press Release—13 Mar. 2000 “Chicago Center for Clinical Research Study suggest New, More Effective Way to Treat Older Women with High Cholesterol”

Clifton-Bligh P B, Baber R J, Fulcher G R, Nery M L, Moreton T. The effect of isoflavones extracted from red clover (Rimostil) on lipid and bone metabolism. Menopause (in submission) 2000.

Evans M P, Fleming K C, Evans J M (1995) Hormone Replacement Therapy: management of common problems Mayo Clin Proc 70(8): 800-5

Gennaro A R (ed.) Remington's Pharmaceutical Sciences Mack Publishing Company, 17th Edition, Easton Pa., USA 1985.

Goodman Gilman A (ed.) Goodman & Gilman's The Pharmacological Basis of Therapeutics, 7th Edition, McGraw Hill Publications, 1985

Hebert P R, Gaziano J M, Chan K S, Hennekens C H (1997) Cholesterol lowering with statin drugs, risk of stroke, and total mortality. An overview of randomized trials. JAMA 278(4): 313-21

Hodgson et al (1998) Supplementation with isoflavonoid phytoestrogens does not alter serum lipid concentrations: a randomised controlled trial in humans. Journal of Nutrition 128: 728-332.

Hulley et al (1998) Randomized trial of estrogen plus progestin for secondary prevention of coronary heart disease in postmenopausal women JAMA 280(7): 605-613; see also related letters JAMA 1999, 281:796-797

Nestel P et al (1997) Soy isoflavones improve systemic arterial compliance but not plasma lipids in menopausal and peri-menopausal women Arteriosclerosis, Thrombosis and Vascular Biology 17: 3392-3398.

Nestel P et al (1999) Isoflavones from red clover improves systemic arterial compliance but not plasma lipids in menopausal women. Journal of Clinical Endocrinology and Metabolism 84: 895-898.

Panchagnula R, Pillai O, Nair V B, Ramrarao P. Transdermal iontophoresis revisited. Curr Opin Chem Biol August 2000; 4(4):468-73

Parfitt K (ed), Martindale 22^{nd} edition—The complete drug reference 1999. Pharmaceutical Press, London

Potter S, Baum J et al (1998) Soy protein and isoflavones: their effects on blood lipids and bone density in postmenopausal women. American Journal of Clinical Nutrition, 68(Suppl):1375S-1379S.

Sacks F M, Pfeffer M A et al (1996) The effect of pravastatin on coronary events after myocardial infarction in patients with average cholesterol levels. Cholesterol and Recurrent Events Trial Investigators. New England Journal of Medicine 335(14):1001-9

Samman S, Lyons Wall P M, Chan G S M, Smith S J, Petocz P (1999) The effect of supplementation with isoflavones on plasma lipids and oxidisability of low density lipoprotein in pre-menopausal women. Atherosclerosis 147: 277-283

Sbarouni E, Kyriakides Z S, Kremastinos D Th (1998) The effect of hormone replacement therapy alone and in combination with simvastatin on plasma lipids of hypercholesterolemic postmenopausal women with coronary artery disease. Journal of the American College of Cardiology 32(5): 1244-50

Scandinavian Simvastatin Survival Study Group (1994) Randomised trial of cholesterol lowering in 4444 patients with coronary heart disease: the Scandinavian Simvastatin Survival Study (4S) Lancet 344:1383-89

Winship K A (1987) Unopposed estrogens. Adverse Drug React Acute Poisoning Rev 6(1): 37-66

Woodside T V, Campbell M J Isoflavones and breast cancer. Journal of the British Menopause Society (in press) 2001

1. A method for regulating circulating lipids levels and/or regulating bone density in a subject comprising the step of:
administering to a subject a pharmaceutically amount of at least one isoflavone or functional derivative, equivalent, or analogue thereof of general formula I in combination with at least one lipid regulating drug.

2. A method of claim 1, wherein said regulating circulating lipid levels is the down-regulation of LDL cholesterol and/or the up-regulation of HDL cholesterol.

3. A method of claim 1 or claim 2, wherein said regulating bone density is the up-regulation of bone density.

4. A method of any one of claims 1 to 3, wherein said isoflavone, or functional derivative, equivalent, or analogue thereof is selected from the compounds of general formulae II-VIII.

5. A method of claim 4, wherein said isoflavone, or functional derivative, equivalent, or analogue thereof is selected from one or more of compounds 1 to 40.

6. A method of claim 5, wherein said isoflavone, or functional derivative, equivalent, or analogue thereof is selected from one or more of formononetin, biochanin, genistein, or daidzein.

7. A method of any one of claims 1 to 6, wherein said lipid regulating drug is selected from the group consisting of a statin, 3-hydroxy-3-methylglutaryl-coenzyme A (HMG-Co A) reductase inhibitors, fibric acid derivatives and related compounds, bile-acid binding resins, nicotinic acid and derivatives thereof, and omega-3 triglycerides.

8. A method of claim 7, wherein said lipid regulating drug is a statin.

9. A method of claim 8, wherein said statin is selected from the group consisting of atorvastatin, cerivastatin, fluvastatin, lovastatin, pravastatin and simvastatin.

10. A method of any one of claims 1 to 3, wherein said isoflavone, or functional derivative, equivalent, or analogue thereof is selected from one or more of formononetin, biochanin, genistein, or daidzein and said lipid regulating drug is atorvastatin or simvastatin.

11. A method of any one of claims 1 to 10, wherein said isoflavone or functional derivative, equivalent or analogue thereof is administered at a rate of 1 mg to 1 g of total isoflavone content per day.

12. A method of claim 11, wherein said isoflavone or functional derivative, equivalent or analogue thereof is administered at a rate of 15 to 50 mg of total isoflavone content per day.

13. A method of any one of claims 1 to 12, wherein said lipid regulating drug is administered at a low dose as defined herein.

14. A method of any one of claims 1 to 13, wherein said at least one isoflavone or functional derivative, equivalent or analogue thereof, and at least one lipid regulating drug are administered simultaneously.

15. A method of any one of claims 1 to 13, wherein said at least one isoflavone or functional derivative, equivalent or analogue thereof and at least one lipid regulating drug are administered sequentially.

16. A method for the treatment of osteoporosis and/or hyperlipidemia comprising the step of:
administering to a subject a pharmaceutically effective amount of at least one isoflavone or functional derivative, equivalent or analogue thereof and at least one lipid lowering drug.

17. A method of claim 16 for the treatment of thrombogenic events, vascular disease, ischaemic heart disease, coronary heart disease and/or arteriosclerosis.

18. A pharmaceutical composition comprising at least one isoflavone, or functional derivative, equivalent, or analogue thereof of general formula I and at least one lipid regulating drug.

19. A composition of claim 18, wherein said isoflavone, or functional derivative, equivalent, or analogue thereof is selected from the compounds of general formulae II-VIII.

20. A composition of claim 19, wherein said isoflavone, or functional derivative, equivalent, or analogue thereof is selected from one or more of compounds 1 to 40.

21. A composition of claim 20, wherein said isoflavone, or functional derivative, equivalent, or analogue thereof is selected from one or more of formononetin, biochanin, genistein, or daidzein.

22. A composition of any one of claims 18 to 21, wherein said lipid regulating drug is chosen from the group consisting of a statin, 3-hydroxy-3-methylglutaryl-coenzyme A (HMG-Co A) reductase inhibitors, fibric acid derivatives and related compounds, bile-acid binding resins, nicotinic acid and-derivatives thereof, and omega-3 triglycerides.

23. A composition of claim 22, wherein said lipid regulating drug is a statin.

24. A composition of claim 23, wherein said statin is selected from the group consisting of atorvastatin, cerivastatin, fluvastatin, lovastatin, pravastatin and simvastatin.

25. A composition of claim 18, wherein said isoflavone, or functional derivative, equivalent, or analogue thereof is selected from one or more of formononetin, biochanin, genistein, or daidzein and said lipid regulating drug is a statin.

26. A composition of any one of claims 18 to 25, wherein said composition further comprises a pharmaceutically acceptable carrier, excipient, auxiliary and/or diluent.

27. A composition of any one of claims 18 to 26, wherein said composition contains between 1 mg and 1 g of the at least one isoflavone or functional derivative, equivalent, or analogue thereof.

28. A composition of claim 27, wherein said composition contains between 10 and 160 mg of the at least one isoflavone or functional derivative, equivalent, or analogue thereof.

29. A composition of claim 27 or claim 28, wherein said composition contains the lipid regulating drug at a concentration suitable for low dose administration.

30. Use of at least one isoflavone or functional derivative, equivalent or analogue thereof of general formula I, at least one lipid regulating drug and a pharmaceutically acceptable carrier and/or diluent in the manufacture of a pharmaceutical composition.

31. Use of at least one isoflavone or functional derivative, equivalent or analogue thereof of general formula I and at least one lipid regulating drug in the manufacture of a medicament for regulating circulating lipids levels and/or regulating bone density.

32. Use of at least one isoflavone or functional derivative, equivalent or analogue thereof of general formula I and at least one lipid regulating drug in the manufacture of a medicament for the treatment of osteoporosis, hyperlipidemia, thrombogenic events, vascular disease, ischaemic heart disease, coronary heart disease and/or arteriosclerosis.

33. A pharmaceutical agent for regulating circulating lipids levels and/or regulating bone density, said agent comprising at least one isoflavone or functional derivative, equivalent or analogue thereof of general formula I and at least one lipid regulating drug

34. A pharmaceutical agent for the treatment of osteoporosis and/or hyperlipidemia, said agent comprising at least one isoflavone or functional derivative, equivalent or analogue thereof of general formula I and at least one lipid regulating drug.
